(19) United States
(12) Patent Application Publication
(10) Pub. No.: US 2025/0099675 A1
(43) Pub. Date: Mar. 27, 2025

(54) INFUSION SET WITH ADHESIVE PATCH LINER

(71) Applicant: Tandem Diabetes Care, Inc., San Diego, CA (US)

(72) Inventors: Amrith Karunaratne, Irvine, CA (US); Justin Coker, Roseville, CA (US); Kennie Hsu, Tustin, CA (US); Hugo Moctezuma, Irvine, CA (US); Amber Stansberry, Irvine, CA (US)

(21) Appl. No.: 18/475,916

(22) Filed: Sep. 27, 2023

(51) Int. Cl. A61M 5/142

(52) U.S. Cl. CPC A61M 5/14248; A61M 2005/14252; A61M 2209/088

(57) ABSTRACT

Disclosed herein are improved infusion sets including adhesive patches with adhesive patch liners configured to reduce folding of the adhesive patch onto itself and liner tearing. Various features can be provided to the path liner to encourage users to grip the liner at a point on the circumference of the adhesive patch and then peel alongside the circumference in a continuous motion.

### TECHNICAL FIELD

The present disclosure relates infusion sets that facilitate injection of medicament into a body of a patient.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type I, or in some cases, type II diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Some portable infusion pumps deliver medicament to patients through infusion sets that include tubing extending from the pump and a cannula with an associated needle that penetrates the patient's skin at an infusion site to allow infusion of the medicament through the cannula and into the patient. If a patient leaves the needle injected at the injection site for too long a period of time, unwanted side effects such as infection and the accumulation of fat and scar tissue can result. Therefore, patients are often instructed to rotate infusion sites to avoid or minimize side effects. Depending on the type of cannula used, the general physiological response of the patient with regard to insulin absorption, and other factors, the time needed between insulin site rotations can vary. Often, sites are rotated every 24-48 hours or every 48-72 hours.

Each time a patient needs to employ a new infusion set, the patient's first interaction with the infusion set is peeling off an adhesive liner patch that covers the adhesive on the underside of the patch that adheres the patch to the user's skin. This interaction can at times cause issues that damage the adhesive patch's functionality. Such issues can include folding of the adhesive patch onto itself and liner tearing, both of which cause reduced areas of adhesive exposure on the user. With less bonding area between the user and the infusion set, the adhesive patch becomes increasingly susceptible to delamination, leading to disruption in infusion therapy and reduced infusion set wear life. In the case of either issue, users may also instead dispose of the infusion set without even using it in order to prevent such risk of disruption. Many infusion sets therefore experience a high percentage of failures. It is therefore important to make the process of peeling of the adhesive patch liner easy to perform without causing any issues of damage to the adhesive patch's functionality.

### SUMMARY

Disclosed herein are improved infusion sets including adhesive patches with adhesive patch liners configured to reduce folding of the adhesive patch onto itself and liner tearing. Various features can be provided to the patch liner to encourage users to grip the liner at a point on the circumference of the adhesive patch and then peel alongside the circumference in a continuous motion.

In an embodiment, an infusion set configured to facilitate delivery of medicament from an infusion pump to a user can include an adhesive patch having an adhesive on an underside of the patch configured to attach that adhesive patch to a body of a user and an opening extending through the underside of the adhesive patch through which a cannula is configured to be inserted into the body of the user. A patch liner can be removably attached to the underside of the patch and include a continuous spline-shaped liner cut extending across a diameter of the adhesive patch to divide the patch liner into a first portion and a second portion.

In an embodiment, an infusion set configured to facilitate delivery of medicament from an infusion pump to a user can include an adhesive patch having an adhesive on an underside of the patch configured to attach that adhesive patch to a body of a user and an opening extending through the underside of the adhesive patch through which a cannula is configured to be inserted into the body of the user. A patch liner can be removably attached to the underside of the patch and include at least one tab configured to be grasped by the user to pull the patch liner off of the adhesive patch. The at least one tab can be oriented such that when the user pulls the tab a force removing the patch liner from the adhesive patch is directed generally along a circumference of the adhesive patch.

In an embodiment, an infusion set configured to facilitate delivery of medicament from an infusion pump to a user can include an adhesive patch having an adhesive on an underside of the patch configured to attach that adhesive patch to a body of a user and an opening extending through the underside of the adhesive patch through which a cannula is configured to be inserted into the body of the user. A patch liner can be removably attached to the underside of the patch and be divided into a separate first portion and second portion. Each of the first portion and the second portion can overlap a diameter of the adhesive patch around a circumference of the adhesive patch.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict an embodiment of an ambulatory infusion pump with an infusion set according to an embodiment of the disclosure.

FIG. 2 depicts an infusion set patch according to an embodiment of the disclosure.

FIGS. 3A-3E depict an infusion set patch according to an embodiment of the disclosure.

FIGS. 4A-4B depict infusion set patch liners according to embodiments of the disclosure.

FIG. 5 depicts infusion set patch liners according to embodiments of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

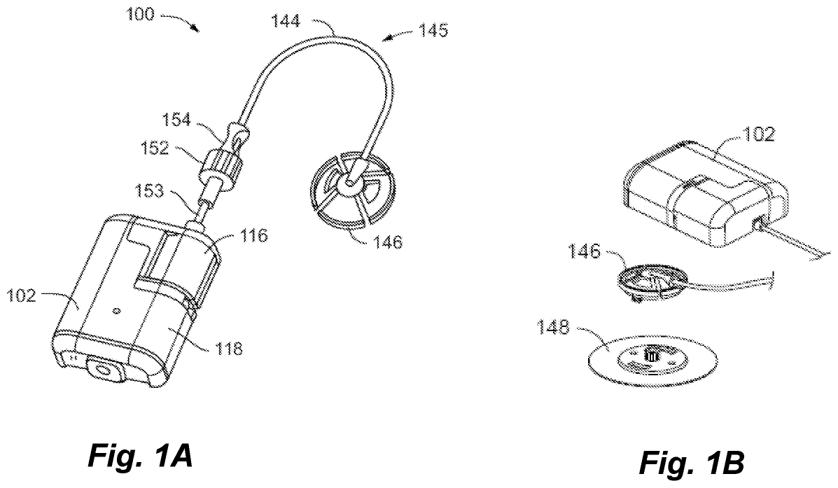

FIGS. 1A-1B depict an exemplary medical device that can be used with embodiments of the disclosure. In this embodiment, the medical device is configured as a pump 102, such as an infusion pump, that can include a pumping or delivery mechanism and reservoir for delivering a medicament to a patient. In one embodiment, the medical device can be a portable pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

As depicted in FIGS. 1A-1B, pump system 100 can include a pump 102 and an infusion set 145. In embodiments, pump system 100 can include a drive unit 118 and a cartridge 116 having a short length of tubing 153 and a connector 152 extending therefrom. Infusion set 145 can include tubing 144 extending between a connector 154 and a site connector 146. Connector 154 on infusion set 145 can be configured to couple to pump 102 at connector 152 of pump 102. As shown in FIG. 1B, site connector 146 can be configured to be attached to an infusion site 148 on a user through which medicament from the pump is delivered to a patient to a cannula extending from the infusion site 148 into the user's skin.

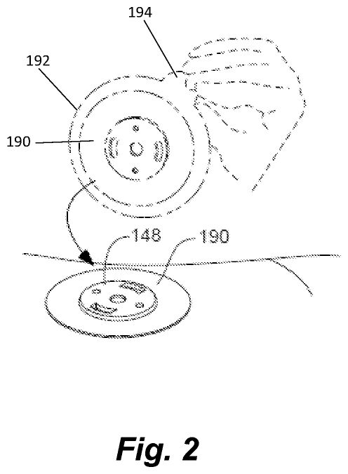

Referring now to FIG. 2, an infusion site 148 including an adhesive patch 190 for adhering the infusion site 148 to the body of a user is depicted. Adhesive patch 190 includes a patch liner 192 that covers the adhesive underside of patch 190 until the user is ready to place the patch 190 on the user's body. In this embodiment, patch liner 192 includes a tab 194 for the user to begin peeling off the liner 192. However, the tab 194 causes the user to pull on a relatively large area of the patch 190 in order to remove the liner 192. As noted above, this can cause issues such as folding of the adhesive patch onto itself and liner tearing that can reduce the effectiveness of the patch or even require the patch to be discarded without being used. In addition, because infusion sets are increasingly being configured to be worn for longer periods of time, stronger adhesives are being used with such infusion sets, which can further exacerbate such problems. Embodiments disclosed herein therefore seek to provide an adhesive patch liner that is easy to remove without causing any issues of damage to the adhesive patch's functionality.

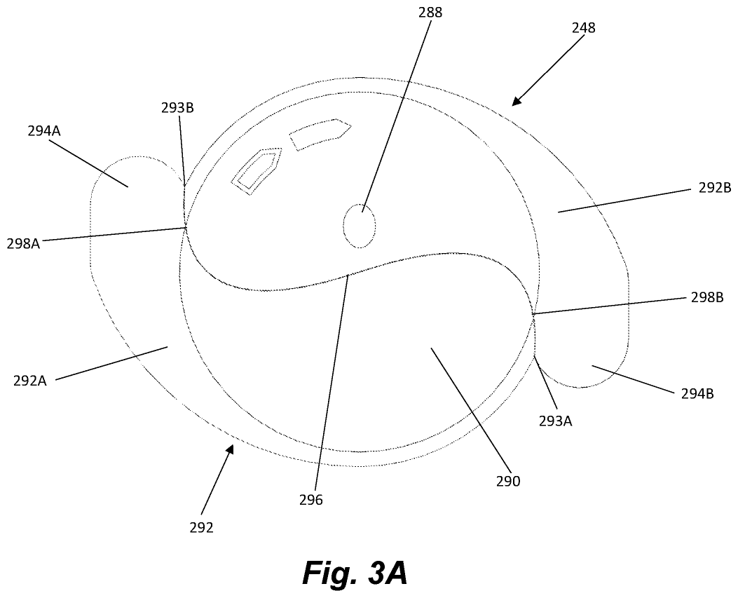

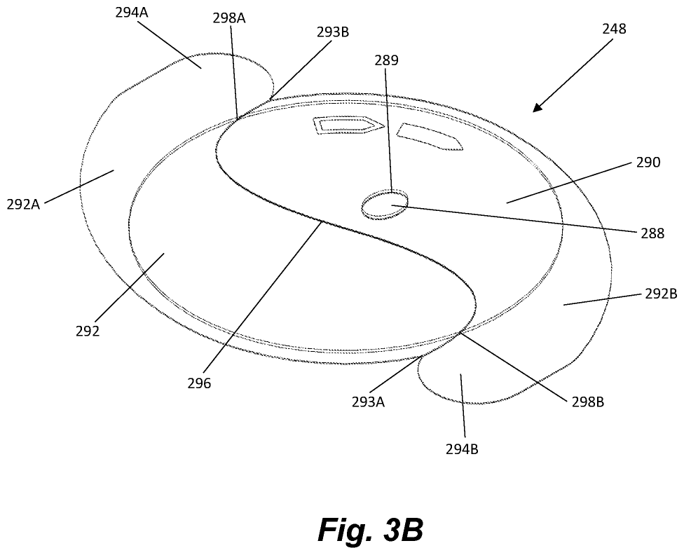

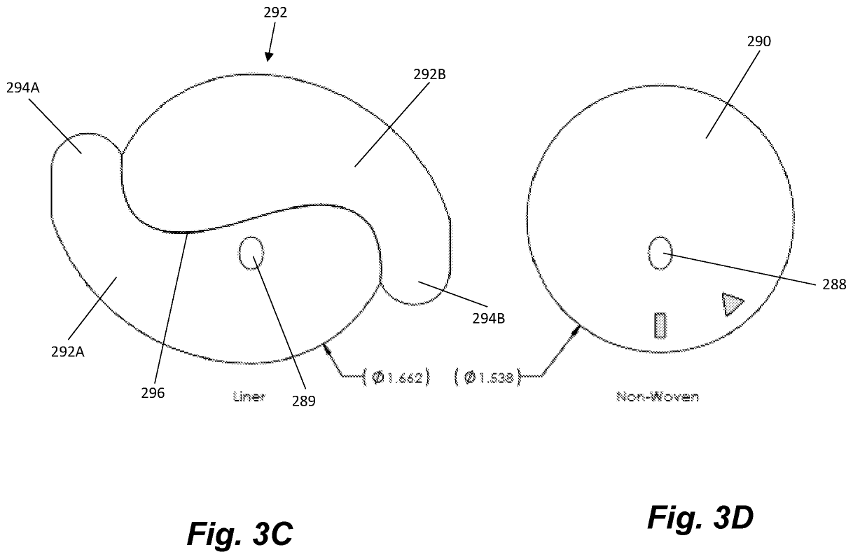

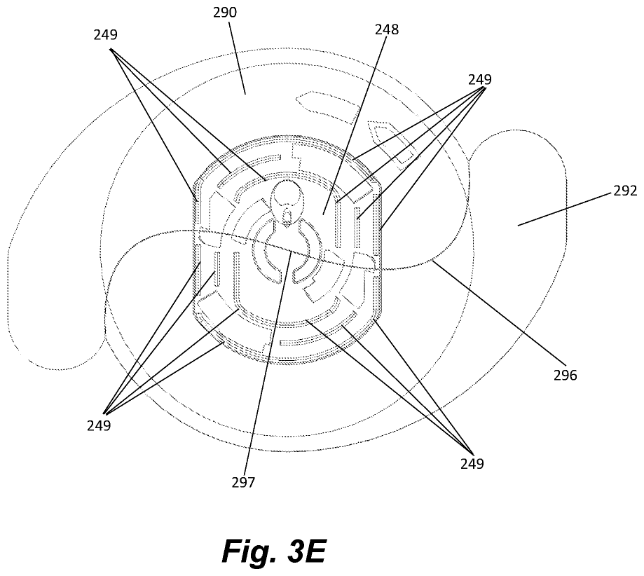

Referring now to FIGS. 3A-3E, an infusion site 248 including an adhesive patch 290 and a patch liner 292 is depicted. Patch liner 292 can be comprised of first 292A and second 292B portions. The portions of patch liner 292 are split via a continuous spline-shaped liner cut 296. Each portion 292A, 292B of patch liner 292 includes a corresponding point-loaded tab 294A, 294B such that patch liner 292 includes a pair of opposing tabs. Each portion 292A,292B can overlap some or all of a diameter of the patch 290 and can arc concentrically around the circumference of patch 290 for at least part of each portion, including at each intersection 293A, 293B between tabs 294A, 294B and the opposing portion 292A, 292B. In this embodiment, the two portions 292A, 292B of patch liner 292 are generally mirror images of one another. The use of point-loaded tabs in conjunction with a spline-shaped liner cut, which arcs in relation to the circumference of the adhesive patch, encourages user to grip the liner at a point on the circumference of the adhesive path and then peel alongside the circumference in a continuous motion.

The depicted design requires the user to begin peeling the liner off of the patch at a small area or “point” (i.e., point-loaded) on the liner where the spline is cut at the edge of the patch. These locations are identified as point 298A and point 298B in FIGS. 3A and 3B. As the user pulls on either tab 294A, 294B, the corresponding liner portion 292A, 292B necessarily disengages from the patch 290 at the single point 298A, 298B. Due to the manner in which each tab 294A, 294B is orientated along the periphery of the patch 290, when the user pulls on the tab 294A, 294B, the force is directed along the periphery or circumference of the patch. Patch portions 292A, 292B increase in width around the circumference of the patch 290 from the initial point 298A, 298B, such that as the user continues to pull a greater amount of liner 292 is removed. This configuration makes it intuitive to peel in a continuous arcing motion in a single direction around the patch, which reduces the effect of folding or bending of the patch in contrast to a tab 192 such as that in FIG. 2 where a user is directed to pull directly across the patch. In addition, when the user is peeling a first patch portion (e.g., 292A), the second patch portion (e.g., 292B) helps the patch 290 remain stable while the first portion is removed.

The patch liner 292 design of FIGS. 3A-3E also provides a continuous peel profile that anchors the point of leverage through the middle area of the adhesive patch due to the spline cut 296 extending across the diameter of the patch through a middle area of the patch. Using the middle area of the patch provides a stronger point of leverage as opposed to relying on points of leverage closer to the circumference of the adhesive patch. This significantly reduces the effect of liner tearing. In addition, the spline provides multiple curves of varying radii that also reduce liner tearing because sharp curves or changes in peel direction that could create different, and easily tearable, leverage points are avoided. The spline cut 296 also avoids the central hole 288 in the patch 290 and corresponding hole 289 in the liner 292 through which a cannula would extend into the user's body, which is another area that could cause tearing of the liner if the cut through the liner were to intersect it.

FIG. 3E illustrates a further feature of patch liner 292. As noted in FIG. 2, the adhesive patch 190 can be attached to an infusion site hub 148 that interfaces with the site connector 146 of the infusion set. The hub 148 is therefore adhered to the patch 190. In some embodiments, an infusion site hub is adhered to a corresponding patch using heat staking, in which the hub has small energy directors that are melted into the patch's non-woven fabric to secure the two components to each other. FIG. 3E depicts these energy directors 249 of an infusion site hub 248 corresponding to patch 290 at which the infusion site hub 248 and patch 290 are adhered together. Applicants have found that if the liner cut 296 intersects a location where the hub 248 is heat staked to the patch 290 (i.e, at an energy director 249), it is more likely that the liner 292 will stick when attempted to be removed and potentially tear at that location. The portion of liner cut 296 indicated by reference number 297 is therefore specifically designed to curve in such a manner so as to avoid overlapping any of the energy directors 249 in order to mitigate the risk of the patch liner 292 tearing.

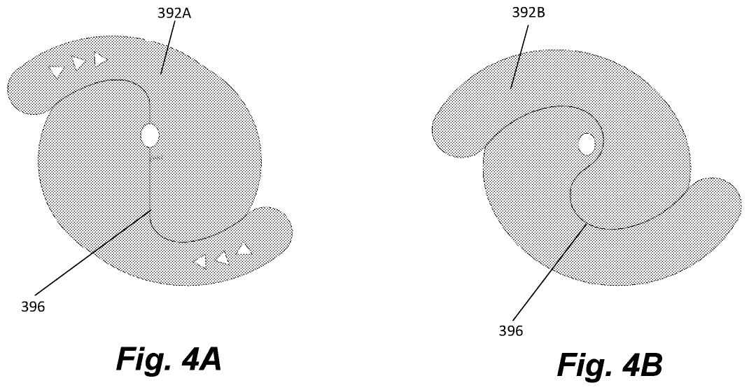

FIGS. 4A-4B depict additional patch liner designs 392A, 392B according to embodiments of the disclosure. Each liner 392 also includes a continuous spline-shaped liner cut 396 that would extend across a diameter of an underlying adhesive patch to divide each liner 392 into two distinct portions. Liner portions 392 would similarly overlap the underlying patch and arc around the patch to facilitate peeling in a continuous arcing motion in a single direction around the patch. Liners 392A and 392B would therefore provide similar advantages to those described above with respect to FIGS. 3A-3C.

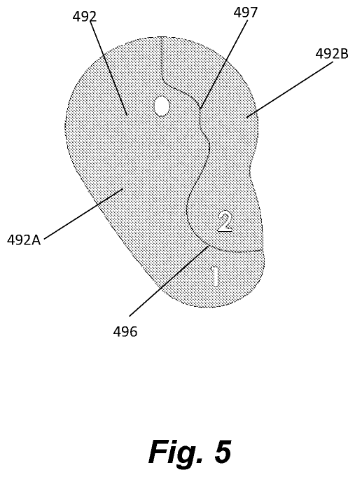

FIG. 5 depicts a patch liner design 492 according to another embodiment of the disclosure. Patch liner 492 includes first 492A and second 492B portions intend to be removed in a specific order, with, in some embodiments a “1” or other indication on first patch liner 492A and a “2” or other indication on second patch liner 492B providing visual instruction to the user as to the intended order of removal. There is also a substantial difference in size or surface area of each of the two portions in contact with the adhesive patch relative to previous embodiments (i.e., first portion 492A is much larger than second portion 492B wherein in prior embodiments the two portions may generally be the same size). This configuration further aids in minimizing the risk of the adhesive patch to fold and stick onto itself during the liner removal process.

By starting with first portion 492A (i.e., tab #1), the user removes a large surface area section of the liner while the remaining second portion 492B (i.e., tab #2) provides enough structural integrity around the perimeter (or initial point of peel) of the adhesive patch to prevent the leading edge from folding on itself. Due to the smaller surface area contact of the second portion 492B, it is much easier to remove it from the adhesive patch following removal of the first portion 492A without the edge wanting to fold. The larger a section of liner is, the more surface area contact it has with the adhesive patch and the more likely it is for the edge to tend to fold on itself. The embodiment of FIG. 5 presents a balance where the second portion 492B is just large enough to provide the support needed for the first portion 492A removal without edge folding, while also being small enough to not encourage the edge fold during its removal after the first portion 492A has been removed. The spline cut 496 of FIG. 5 also includes a “jogging” or wave section 497 specifically including in order to avoid any of the heat staking areas between an infusion site hub and adhesive patch corresponding to liner 492 to mitigate the risk of liner tearing, as discussed above.

In embodiments, an infusion set configured to facilitate delivery of medicament from an infusion pump to a user can include an adhesive patch having an adhesive on an underside of the patch configured to attach that adhesive patch to a body of a user and an opening extending through the underside of the adhesive patch through which a cannula is configured to be inserted into the body of the user. A patch liner can be removably attached to the underside of the patch and include a continuous spline-shaped liner cut extending across a diameter of the adhesive patch to divide the patch liner into a first portion and a second portion.

In some embodiments, the continuous spline-shaped liner cut does not intersect the opening extending through the underside of the adhesive patch.

In some embodiments, the first portion and the second portion of the patch liner each include a tab configured to be grasped by the user to pull the corresponding portion off of the adhesive patch.

In some embodiments, each tab is aligned such that when the user pulls the tab the corresponding portion of the patch liner initially separates from the adhesive patch at a single point on the adhesive patch.

In some embodiments, the first portion and the second portion of the patch liner increase in width from the single point around a circumference of the adhesive patch.

In some embodiments, each tab is aligned such that when the user pulls the tab a force removing the corresponding portion of the patch liner is directed generally along a circumference of the adhesive patch.

In some embodiments, the first portion and second portion of the patch liner each overlap the diameter of the adhesive patch.

In some embodiments, at least a portion of each of the first portion and the second portion of the patch liner that overlaps the diameter of the adhesive patch is concentrically aligned with a circumference of the adhesive patch.

In embodiments, an infusion set configured to facilitate delivery of medicament from an infusion pump to a user can include an adhesive patch having an adhesive on an underside of the patch configured to attach that adhesive patch to a body of a user and an opening extending through the underside of the adhesive patch through which a cannula is configured to be inserted into the body of the user. A patch liner can be removably attached to the underside of the patch and include at least one tab configured to be grasped by the user to pull the patch liner off of the adhesive patch. The at least one tab can be oriented such that when the user pulls the tab a force removing the patch liner from the adhesive patch is directed generally along a circumference of the adhesive patch.

In some embodiments, the at least one tab is aligned such that when the user pulls the tab patch liner initially separates from the adhesive patch at a single point on the adhesive patch.

In some embodiments, the patch liner includes a separate first portion and second portion, and the at least one tab includes a first tab on the first portion and a second tab on the second portion.

In some embodiments, the first portion and the second portion of the patch liner increase in width around a circumference of the adhesive patch.

In some embodiments, the first and second portions are separated by a continuous spline-shaped liner cut extending across a diameter of the adhesive patch.

In some embodiments, the continuous spline-shaped liner cut does not intersect the opening extending through the underside of the adhesive patch.

In some embodiments, a body portion of the patch liner connected to the tab overlaps a diameter of the adhesive patch around the circumference of the adhesive patch.

In embodiments, an infusion set configured to facilitate delivery of medicament from an infusion pump to a user can include an adhesive patch having an adhesive on an underside of the patch configured to attach that adhesive patch to a body of a user and an opening extending through the underside of the adhesive patch through which a cannula is configured to be inserted into the body of the user. A patch liner can be removably attached to the underside of the patch and be divided into a separate first portion and second portion. Each of the first portion and the second portion can overlap a diameter of the adhesive patch around a circumference of the adhesive patch.

In some embodiments, at least a portion of each of the first portion and the second portion of the patch liner that overlaps the diameter of the adhesive patch is concentrically aligned with the circumference of the adhesive patch.

In some embodiments, the first portion and the second portion of the patch liner increase in width around the circumference of the adhesive patch.

In some embodiments, the first and second portions are separated by a continuous spline-shaped liner cut extending across a diameter of the adhesive patch.

In some embodiments, the continuous spline-shaped liner cut does not intersect the opening extending through the underside of the adhesive patch.

Although the infusion pump embodiments herein are specifically described primarily with respect to the delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patent application Ser. Nos. 17/368,968; 17/896,492; 18/011,060; 18/071,814; 18/071,835; 18/075,029; 18/090,788 18/115,316; and Ser. No. 18/139,391.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An infusion set configured to facilitate delivery of medicament from an infusion pump to a user, comprising:
an adhesive patch having an adhesive on an underside of the patch configured to attach that adhesive patch to a body of a user and an opening extending through the underside of the adhesive patch through which a cannula is configured to be inserted into the body of the user; and
a patch liner removably attached to the underside of the patch, the patch liner including a continuous spline-shaped liner cut extending across a diameter of the adhesive patch to divide the patch liner into a first portion and a second portion.

2. The infusion set of claim 1, wherein the continuous spline-shaped liner cut does not intersect the opening extending through the underside of the adhesive patch.

3. The infusion set of claim 1, wherein the first portion and the second portion of the patch liner each include a tab configured to be grasped by the user to pull the corresponding portion off of the adhesive patch.

4. The infusion set of claim 3, wherein each tab is aligned such that when the user pulls the tab the corresponding portion of the patch liner initially separates from the adhesive patch at a single point on the adhesive patch.

5. The infusion set of claim 4, wherein the first portion and the second portion of the patch liner increase in width from the single point around a circumference of the adhesive patch.

6. The infusion set of claim 3, wherein each tab is aligned such that when the user pulls the tab a force removing the corresponding portion of the patch liner is directed generally along a circumference of the adhesive patch.

7. The infusion set of claim 1, wherein the first portion and second portion of the patch liner each overlap the diameter of the adhesive patch.

8. The infusion set of claim 7, wherein at least a portion of each of the first portion and the second portion of the patch liner that overlaps the diameter of the adhesive patch is concentrically aligned with a circumference of the adhesive patch.

9. An infusion set configured to facilitate delivery of medicament from an infusion pump to a user, comprising:
an adhesive patch having an adhesive on an underside of the patch configured to attach that adhesive patch to a body of a user and an opening extending through the underside of the adhesive patch through which a cannula is configured to be inserted into the body of the user; and
a patch liner removably attached to the underside of the patch, wherein the patch liner include at least one tab configured to be grasped by the user to pull the patch liner off of the adhesive patch, and wherein the at least one tab is oriented such that when the user pulls the tab a force removing the patch liner from the adhesive patch is directed generally along a circumference of the adhesive patch.

10. The infusion set of claim 9, wherein the at least one tab is aligned such that when the user pulls the tab patch liner initially separates from the adhesive patch at a single point on the adhesive patch.

11. The infusion set of claim 9, wherein the patch liner includes a separate first portion and second portion, and the at least one tab includes a first tab on the first portion and a second tab on the second portion.

12. The infusion set of claim 11, wherein the first portion and the second portion of the patch liner increase in width around a circumference of the adhesive patch.

13. The infusion set of claim 11, wherein the first and second portions are separated by a continuous spline-shaped liner cut extending across a diameter of the adhesive patch.

14. The infusion set of claim 13, wherein the continuous spline-shaped liner cut does not intersect the opening extending through the underside of the adhesive patch.

15. The infusion set of claim 9, wherein a body portion of the patch liner connected to the tab overlaps a diameter of the adhesive patch around the circumference of the adhesive patch.

16. An infusion set configured to facilitate delivery of medicament from an infusion pump to a user, comprising:
an adhesive patch having an adhesive on an underside of the patch configured to attach that adhesive patch to a body of a user and an opening extending through the underside of the adhesive patch through which a cannula is configured to be inserted into the body of the user; and
a patch liner removably attached to the underside of the patch, wherein the patch liner is divided into a separate first portion and second portion, wherein each of the first portion and the second portion overlaps a diameter of the adhesive patch around a circumference of the adhesive patch.

17. The infusion set of claim 16, wherein at least a portion of each of the first portion and the second portion of the patch liner that overlaps the diameter of the adhesive patch is concentrically aligned with the circumference of the adhesive patch.

18. The infusion set of claim 16, wherein the first portion and the second portion of the patch liner increase in width around the circumference of the adhesive patch.

19. The infusion set of claim 16, wherein the first and second portions are separated by a continuous spline-shaped liner cut extending across a diameter of the adhesive patch.

20. The infusion set of claim 19, wherein the continuous spline-shaped liner cut does not intersect the opening extending through the underside of the adhesive patch.
